(19) United States
(12) Patent Application Publication
(10) Pub. No.: US 2025/0054602 A1
(43) Pub. Date: Feb. 13, 2025

(54) FILE INTEGRITY AUDITING FOR MALWARE DETECTION

(71) Applicant: GE Precision Healthcare LLC, Waukesha, WI (US)

(72) Inventors: Nivedha Srinivasan, Bengaluru (IN); Khaleel Ahamad Nadaf, Bengaluru (IN); Tejas Soali, Bengaluru (IN)

(21) Appl. No.: 18/446,304

(22) Filed: Aug. 8, 2023

(51) Int. Cl. G16H 30/20; G06F 21/56

(52) U.S. Cl. CPC G16H 30/20; G06F 21/56; G06F 2221/034

(57) ABSTRACT

A medical imaging device includes memory storing instructions and a classification model. The medical imaging device also includes a processor communicatively coupled to the memory. The instructions, when executed by the processor, cause the medical imaging device to perform actions. The actions include implementing an imaging-related functionality. The actions also include detecting a change event that modifies a memory state of the memory. The actions further include validating the change event based on a comparison between the change event and a baseline representation of the memory state. The actions also include updating an integrity state of the medical imaging device based on the comparison.

### TECHNICAL FIELD

The subject matter disclosed herein relates to computer security and, more particularly, to a system and a method of file integrity auditing for malware detection.

### BACKGROUND

Cyber-attack incidents are becoming increasingly common. Cyber-attack incidents have increased at a larger rate in the healthcare sector relative to other sectors. Cyber-attacks generally involve malicious software or malware entering a networked environment via unauthorized access at a node of the networked. Upon entering the networked environment, malware can quickly spread to other nodes of the networked environment via privilege escalation, code replication, and the like. In any sector, malware can be used to steal proprietary information, disrupt network operations, and the like. In the healthcare sector, malware can substantially disrupt patient care with life-threatening consequences.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a medical imaging device includes memory storing instructions and a classification model. The medical imaging device also includes a processor communicatively coupled to the memory. The instructions, when executed by the processor, cause the medical imaging device to perform actions. The actions include implementing an imaging-related functionality. The actions also include detecting a change event that modifies a memory state of the memory. The actions further include validating the change event based on a comparison between the change event and a baseline representation of the memory state. The actions also include updating an integrity state of the medical imaging device based on the comparison.

In another embodiment, a computer-implemented method of file integrity auditing for malware detection includes detecting, by a processor of a medical imaging device, a change event that modifies a memory state of the medical imaging device. The computer-implemented method also includes validating, by the processor, the change event based on a comparison between the change event and a baseline representation of the memory state. The computer-implemented method also includes updating, by the processor, an integrity state of the medical imaging device based on the comparison.

In a further embodiment, a non-transitory computer-readable medium is provided. The computer-readable medium includes processor-executable code that when executed by a processor, causes the processor to perform actions. The actions include detecting a change event that modifies a memory state of a computing device. The actions also include providing file data characterizing the change event as input to a classification model that is trained using a baseline representation of the memory state. The actions also include receiving a classification and a risk score for the change event at an output of the classification model. The actions also include updating an integrity state of the computing device based on the classification and the risk score.

### BRIEF DESCRIPTION OF THE DRAWINGS

These and other features, aspects, and advantages of the present disclosed subject matter will become better understood when the following detailed description is read with reference to the accompanying drawings in which like characters represent like parts throughout the drawings, wherein:

FIG. 1 is a pictorial representation of a CT imaging system, in accordance with aspects of the present disclosure;

FIG. 2 is a block diagram of the CT imaging system in FIG. 1, in accordance with aspects of the present disclosure;

FIG. 3 is a block diagram of an operating environment of the CT imaging system in FIG. 1, in accordance with aspects of the present disclosure;

FIG. 4 is a flowchart that illustrates a development process for implementing file integrity auditing using machine learning techniques, in accordance with aspects of the present disclosure;

FIG. 5 is a schematic diagram of an example conditional indicator set, in accordance with aspects of the present disclosure;

FIG. 6 is a flowchart that illustrates a method of file integrity auditing for malware detection, in accordance with aspects of the present disclosure; and

FIG. 7 is a schematic diagram of an example error log data structure, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The present disclosure provides systems and methods for file integrity auditing for malware detection, such as in a health care environment. In particular, the disclosed techniques include detecting a change event that modifies a memory state of a computing device, validating the change event based on a comparison between the change event and a baseline representation of the memory state, and updating an integrity state of the computing device based on the comparison. In certain embodiments, detecting a change event includes continuously monitoring file system data that characterizes a memory state of a computing device.

A number of different types of events may serve as change events that modify a memory state of a computing device. For example, file operations (e.g., move, copy, edit, delete, and update) implemented on file system entities (e.g., files, folders, directories, and processes) may modify file system data that characterizes a memory state of a computing device. Such file system data may include file data residing in primary memory of a computing device and secondary memory of the computing device. A difference may exist between file system data prior to implementing a file operation and file system data after implementing the file operation. File data representing that difference may characterize a change event corresponding to the file operation. Examples of change events characterized by such file data may include deviations in file system entities with respect to content, size, access permission, location, and the like.

Change events that modify a memory state of a computing device in a manner that is consistent with a baseline representation of the memory state may be benign change events. For example, a file operation initiated by an application or user to implement a desired functionality of a computing device may be a benign change event. Change events that modify a memory state of a computing device in a manner that is inconsistent with a baseline representation of the memory state may be malicious change events. For example, a file operation initiated by malware to implement an undesired functionality or a functionality that is unrelated to desired functionality of a computing device may be a malicious change event.

In certain embodiments, a classification model may be trained to distinguish between benign change events and malicious change events. For example, a classification model may be trained using a baseline representation of a memory state and/or a set of conditional indicators (conditional indicator set). The baseline representation of the memory state may be created using file data obtained from a file system of a computing device in a reference configuration. The conditional indicator set may define different change events to be audited and validated for malware detection. In certain embodiments, validating a change event includes providing file data that characterizes the change event as input to a classification model. In certain embodiments, validating a change event includes receiving a malicious classification or a benign classification at an output of a classification model based on file data that characterizes the change event. In certain embodiments, validating a change event includes receiving a risk metric for the change event at an output of a classification model based on file data that characterizes the change event. The risk metric may estimate a likelihood that a malware operation initiated the change event.

While aspects of the following discussion may be provided in the context of medical imaging, it should be appreciated that the present techniques are not limited to medical imaging contexts. Indeed, the provision of examples and explanations in the context of medical imaging is only to facilitate explanation by providing instances of real-world implementations and applications. However, the present approaches may also be utilized in any computing context that involves file integrity auditing for malware detection.

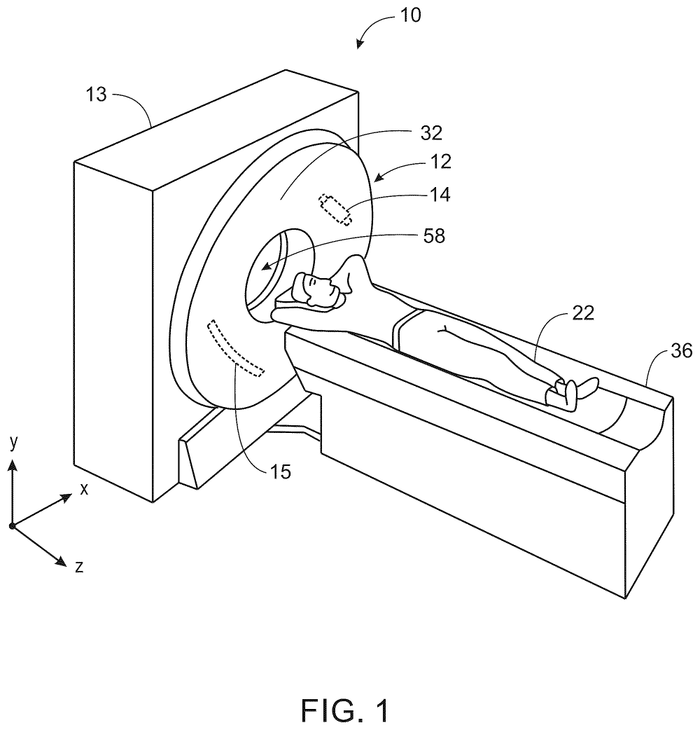

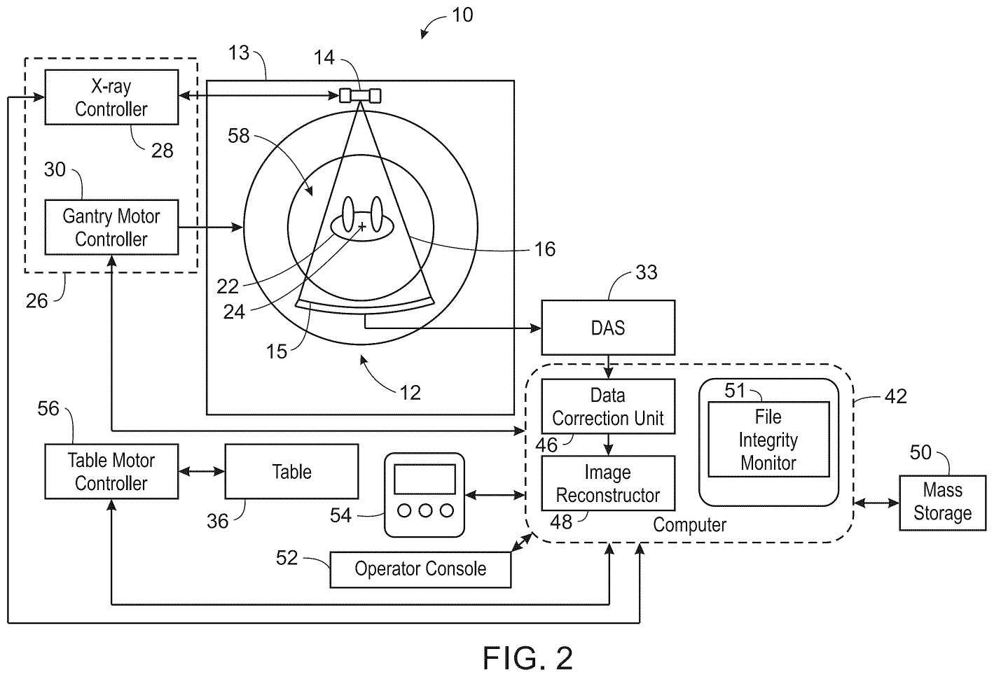

With the preceding in mind and referring to FIGS. 1 and 2, a CT imaging system 10 is shown, by way of example. As may be appreciated, the CT imaging system 10 disclosed and described with respect to FIGS. 1 and 2 merely provides a real-world example of the type of processor-enabled or functionalized device that may be present in a health care environment and that, due to the incorporation of processor-enabled and/or computer-implemented functionality, may be subject to malware and/or other malicious electronic attack. As may be appreciated, in a hospital or clinical context, numerous types of such electronic devices (e.g., imaging devices, patient monitors, diagnostic equipment, therapeutic equipment, and so forth) of different types, ages, manufacture and so forth may be present, may communicate or be networked together, and must be supported. However, the diversity of such devices may make rigorous monitoring and/or implementation of a uniform or out-of-the-box (OOB) type security system impractical. With this in mind, FIGS. 1 and 2 describe one such system to illustrate both a real-world example of a possible target system and the complexity that may be involved in such a system that distinguishes such medical-type systems from what might be found in an office or other non-medical context.

With this in mind, and turning to the figures, the CT imaging system 10 (e.g., CT scanner) includes a gantry 12 having a housing 13 (e.g., gantry housing) and a rotating component and a stationary component. By way of example, the gantry 12 has an X-ray source 14 that projects a beam of X-rays 16 toward an X-ray detector assembly or X-ray detector array 15 (e.g., having a plurality of detector modules) on the opposite side of the gantry 12. The X-ray detector assembly 15 is coupled to data acquisition systems (DAS) 33.

The plurality of detector modules of the X-ray detector assembly 15 detect the projected X-rays that pass through a patient or subject 22, and DAS 33 converts the data to digital signals for subsequent processing. During a scan to acquire X-ray projection data, gantry 12 and the components mounted thereon rotate about a center of rotation 24 (e.g., isocenter) so as to allow volumetric imaging. Rotation of gantry 12 and the operation of X-ray source 14 are governed by a control mechanism 26 of CT system 10. Control mechanism 26 includes an X-ray controller 28 that provides power and timing signals to the X-ray source 14 and a gantry motor controller 30 that controls the rotational speed and position of gantry 12.

A computer 42 (separate from or a part of the CT imaging system 10) includes a data correction unit 46 for processing or correcting the CT scan data from the DAS 33. The computer 42 also includes an image reconstructor 48. The image reconstructor 48 receives sampled and digitized X-ray data from DAS 33 and performs high-speed reconstruction. The reconstructed image is applied as an input to the computer 42, which stores the image in a mass storage device 50. Computer 42 also receives commands and scanning parameters from an operator via console 52. An associated display 54 allows the operator to observe the reconstructed image and other data from the computer 42. The operator supplied commands and parameters are used by computer 42 to provide control signals and information to the DAS 33, X-ray controller 28, and gantry motor controller 30. In addition, computer 42 operates a table motor controller 56, which controls a motorized table 36 to position the patient 22 relative to the gantry 12. Particularly, table 36 moves portions of the patient 22 through a gantry opening or bore 58. With the preceding in mind, it may be seen that such a system includes many computational or processor-based aspects that may be subject to compromise by a malicious actor. Indeed, the complexity, specific functionality provided, and networked nature of such a device may make conventional approaches to security infeasible or impractical.

With this in mind, and in accordance with the present disclosure, the computer 42 further includes a file integrity monitor 51. The file integrity monitor 51 is generally configured to detect change events that modify a memory state of the CT imaging system 10 (or other protected device, medical or otherwise). The file integrity monitor 51 is further configured to validate a change event based on a comparison between the change event and a baseline representation of the memory state. Based on the comparison, the file integrity monitor 51 is further configured to update an integrity state of the CT imaging system 10.

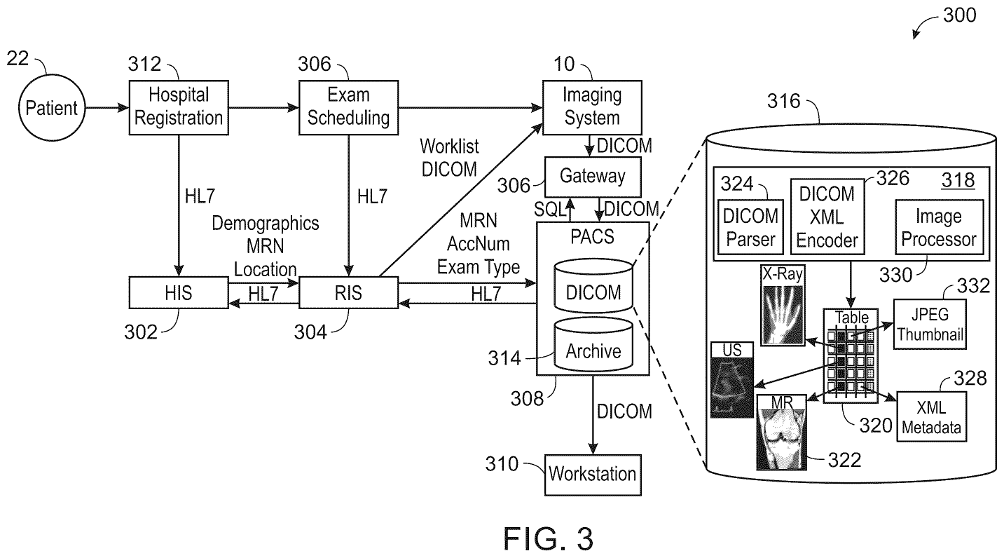

FIG. 3 is a block diagram of an example operating environment 300 for the CT imaging system 10, in accordance with aspects of the present disclosure. The operating environment 300 generally illustrates dataflow between different information systems in a healthcare environment and further illustrates the complexity and risks of such an environment to a malicious attack. The operating environment 300 includes the CT imaging system 10 or other medical imaging, monitoring, or diagnostic equipment. The operating environment 300 also includes a hospital information system (HIS) 302, a radiology information system (RIS) 304, a gateway 306, a picture archiving and communication system (PACS) 308, and a workstation 310. As may be appreciated from the discussion below, much of the data and information exchanged in such an environment may be personal in nature (including personal identifiable information (PII), health or medical information, financial information, and so forth). Likewise, as may be evident from the data and work flow discussion below, the complexity of such systems in a health care environment may be disrupted via malicious attack, thereby impacting the integrity of patient and facility data, the ability to provide treatment or services, and potentially impacting the safety and well-being of patients.

With this in mind, and by way of one real-world example, prior to lying down on the table 36 of the CT imaging system 10 for a scan, the patient 22 completes a registration process 312 and an exam scheduling process 314 with the healthcare environment. The registration process 312 involves creating or updating an electronic medical record (EMR) at the HIS 302. The EMR includes an identifier such as a medical record number (MRN) that uniquely associates the EMR with the patient 22 in the healthcare environment. As part of the registration process 312, a Health Layer Seven (HL7) message with patient demographic data (e.g., patient name, birth date, contact information) and can be sent to the HIS 302. The HL7 message sent to the HIS 302 can also include a location of the patient 22 within the healthcare environment.

The exam scheduling process 314 involves creating a study order for the scan via HL7 messaging with the RIS 304. The RIS 304 can obtain patient demographic data and the identifier of the EMR associated with the patient 22 for the study order via an HL7 message exchange with the HIS 302. As part of the exam scheduling process 314, the study order can be populated with exam specific information, such as imaging modality (e.g., CT), exam or study description (e.g., anatomical region being imaged), and an identifier (e.g., an examination accession number (AccNum) that uniquely identifies the examination (e.g., the CT scan of the patient 22 being scheduled) in the healthcare environment.

The CT imaging system 10 can send one or more Digital Imaging and Communications in Medicine (DICOM) standard files to the PACS 308 via the gateway 306 after completing the scan of the patient 22. Each DICOM file can include image data generated by the CT imaging system 10 for the scan. Each DICOM file sent by the CT imaging system 10 can also include metadata (e.g., DICOM tags) that is specific to the scan. Metadata in a DICOM file can include patient demographic data (e.g., patient name), an EMR identifier (e.g., MRN), exam specific information (e.g., study description, study time/date, imaging modality, and/or examination identifier).

A DICOM database 316 of the PACS 308 can receive DICOM files sent by the CT imaging system 10 for the scan of the patient 22. An image server 318 of the DICOM database 316 can process the DICOM files sent by the CT imaging system 10 for storage in a data structure such as table 320. The image server 318 can store image data 322 from the DICOM files sent by the CT imaging system 10 in a column of the table 320. Another column of the table 320 can include metadata from the DICOM files sent by the CT imaging system 10. To that end, DICOM parser 324 of the image server 318 can extract metadata from the DICOM files into an in-memory structure. DICOM Extensible Markup Language (XML) encoder 326 can convert the in-memory structure with the extracted metadata into an XML document 328.

The PACS 308 can provide the workstation 310 with access to the table 320 responsive to a query sent by the workstation 310. The workstation 310 can send a radiologist report to the PACS 308 that includes textual data describing results of the scan. The radiologist report can also include a key image from the image data 322 and radiologist measurement data. An image processor 330 of the image server 318 can extract a subset of the image data 322 that corresponds to the key image. The image processor 330 can convert the subset of the image data 322 that corresponds to the key image into a thumbnail representation 332 of the key image having a different image format (e.g., Joint Photographic Experts Group (JPEG) image format). The image processor 330 can store the thumbnail representation 332 of the key image in another column of the table 320.

Returning to FIGS. 1 and 2, the computer 42 includes processing circuitry. The processing circuitry may be one or more general or application-specific microprocessors. The processing circuitry can be configured to execute instructions stored in a memory to perform various actions. For example, the processing circuitry can be utilized for detecting a change event that modifies a memory state of the CT imaging system 10, validating the change event based on a comparison between the change event and a baseline representation of the memory state, and updating an integrity state of the CT imaging system 10 based on the comparison.

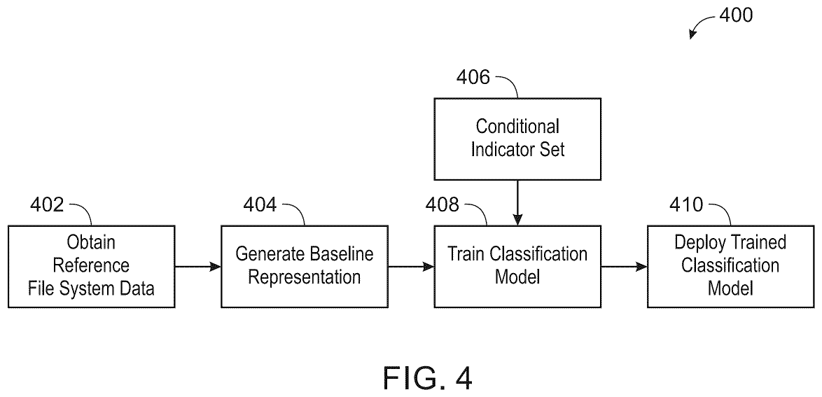

FIG. 4 is a flowchart that illustrates a development process 400 for implementing file integrity auditing using machine learning techniques, in accordance with aspects of the present disclosure and which may be used with or on medical or other devices, such as, but not limited to, those described with respect to the preceding figures. In particular, FIG. 4 illustrates various steps involved in developing and training a classification model for file integrity auditing using supervised learning techniques. In certain embodiments, the classification model developed by the process 400 can be used by the file integrity monitor 51 of FIG. 2 for malware detection.

At block 402, the process 400 includes obtaining reference file system data for a computing device (e.g., the CT imaging system 10). File system data generally refers to system configuration information that provides a complete or substantially complete representation of computing device software. File system data generally includes system data and application data. System data can include file data comprising an operating system and extensions of the operating system, such as initialization data, configuration data, library data, and the like. Application data can include file data comprising a computing program that executes within a runtime environment provided by an operating system to perform specific tasks. File system data can be stored in primary or main memory (e.g., in-memory data structures) of a computing device. Examples of file data stored in primary or main memory can include cache data, a process table, an open file table (e.g., process specific and/or system wide), a directory structure cache, a mount table, and the like. File system data can also be stored in secondary memory (e.g., in-disk data structures) of a computing device. Examples of file data stored in secondary memory include a file control block, a page file, a boot control block, a volume control block, and the like.

At block 404, the process 400 also includes generating a baseline representation of a memory state of a computing device (e.g., the CT imaging system 10) using the reference file system data. In certain embodiments, the baseline representation can be generated using reference file system data obtained from a single computing device. In certain embodiments, the baseline representation can be generated using reference file system data obtained from multiple computing devices having the same or a substantially similar reference configuration.

At block 406, the process 400 also includes obtaining a conditional indicator set. Each conditional indicator of a conditional indicator set can evaluate different variances or different sets of variances that modify a memory state of a computing device. Examples of such variances include: file content being updated to encrypted data; intermittent file content encryption; protected health information (PHI) data being accessed by users and/or services; file-level permission changes or privilege escalation; folder or directory-level permission changes or privilege escalation; file modifications by users and/or services; file transfer operations involving peripheral devices; file content deviations; file renaming, deletion, and/or movement; orphaned software, folders, and/or files; out-of-boundary file types; orphaned processes in a running state; and/or other variances that modify a memory state of a computing device.

At block 408, the process 400 also includes training a classification model with the baseline representation and/or the conditional indicator set using supervised learning techniques. Supervised learning techniques can include: linear regression, logistic regression, neural networks, support vector machines, naive Bayes, random forest, classification trees, and the like. Supervised learning techniques generally refer to machine learning processes that use labeled training datasets or labeled training data to train models such as classification models to classify input data or predict outcomes. Training datasets generally include numerous instances or examples of input data. Each instance included in the training dataset can be described by a number of features or attributes. Each instance of labeled training dataset includes a class label that defines a particular class (e.g., malicious or benign) to which that instance belongs. In certain embodiments, the baseline representation generated at block 404 and the conditional indicator set obtained at block 406 can be used to compile a labeled dataset for training the classification model at block 408. Each instance of the labeled dataset for training the classification model includes file data characterizing a change event and a class label (e.g., a malicious class label or a benign class label). In certain embodiments, the classification model can be a k-nearest neighbor (KNN) model that classifies unseen instances of input data based on similarity measurements.

At block 410, the process 400 also includes deploying the trained classification model. In certain embodiments, the trained classification model can be deployed using a self-contained application package that includes functionality for ingesting, processing, and/or storing file data characterizing change events. The self-contained application package can be stored in a memory of a computing device (e.g., the CT imaging system 10 or other medical or non-medical processor-based system) such that the self-contained application package is isolated from other file data in the memory. In certain embodiments, the trained classification model can be directly deployed in file system data of a computing device. For example, the trained classification model can be included in program instructions that, when executed by a processor of a computing device, cause the computing device to implement a desired functionality (e.g., an imaging-related functionality, such as generating image data, maintaining image data, manipulating image data, formatting image data, and/or modifying metadata of a DICOM file).

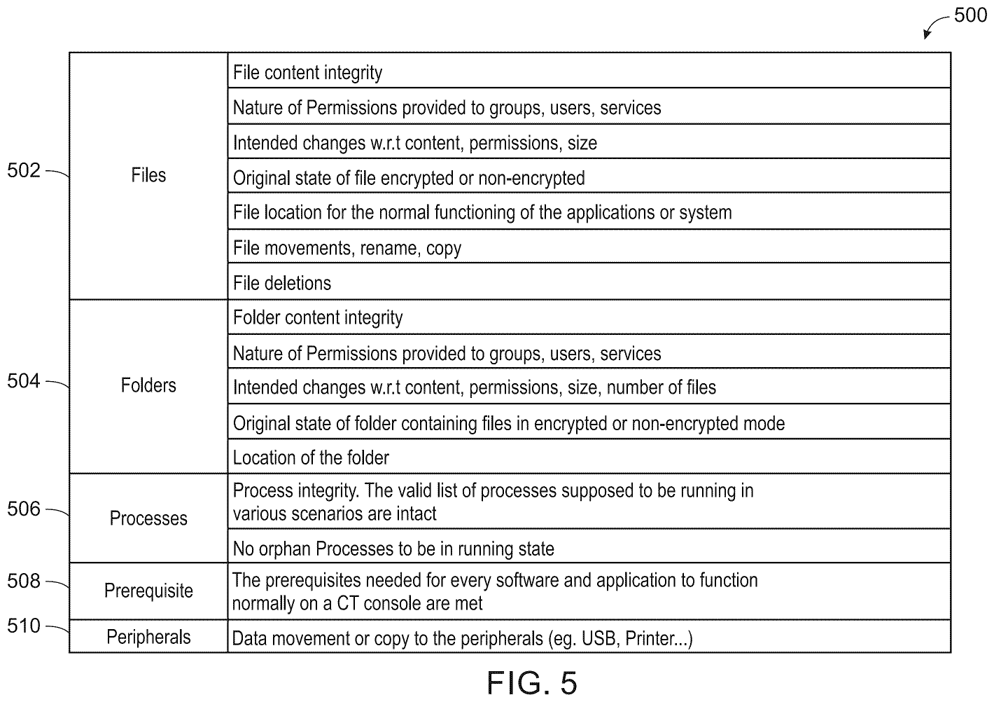

FIG. 5 is a schematic diagram of an example conditional indicator set 500, in accordance with aspects of the present disclosure. The conditional indicator set 500 includes a file indicator subset 502, a folder indicator subset 504, a process indicator subset 506, a prerequisite indicator subset 508, and a peripheral device subset 510. The file indicator subset 502 can include conditional indicators that evaluate file-level variances that modify a memory state of a computing device. For example, a conditional indicator within the file indicator subset 502 can evaluate file-level variances in a file control block or an access control list of the computer 42. File-level variances can involve an attribute or content of a file having a value that is different from a corresponding value of that attribute or content in a baseline representation of the memory state. Examples of such file-level variances are shown in FIG. 5 with respect to the file indicator subset 502.

The folder indicator subset 504 can include conditional indicators that evaluate folder-level or directory-level variances that modify a memory state of a computing device. For example, a conditional indicator within the folder indicator subset 504 can evaluate folder-level or directory-level variances in a file control block or an access control list of the computer 42. Folder-level variances can involve an attribute or content of a folder or directory having a value that is different from a corresponding value of that attribute or content in a baseline representation of the memory state. Examples of such folder-level variances are shown in FIG. 5 with respect to the folder indicator subset 504.

The process indicator subset 506 can include conditional indicators that evaluate process-level variances that modify a memory state of a computing device. For example, a conditional indicator within the process indicator subset 506 can evaluate variances in a process control block associated with the image reconstructor 48 or other comparable module or processing block unique to or specific to a processor-based system, such as aa medical imaging system, medical monitoring system, therapy or drug delivery system, and so forth. Another example, a conditional indicator within the process indicator subset 506 can evaluate a process table of the computer 42 to identify an orphan process that remains running after termination of a parent process. Process-level variances can involve an attribute of a process having a value that is different from a corresponding value of that attribute in a baseline representation of the memory state. Examples attributes of such process-level variances are shown in FIG. 5 with respect to the process indicator subset 506.

The prerequisite indicator subset 508 can include conditional indicators relating to availability of requisite hardware or software resources to support functioning of medical device software. For example, a conditional indicator within the prerequisite indicator subset 508 can evaluate whether sufficient hardware or software resources are available within the CT imaging system 10 to support normal operation of the image reconstructor 48 or similar clinical processing block or module. The peripheral device subset 510 can include conditional indicators relating to interactions with peripheral devices. For example, a conditional indicator within the prerequisite indicator subset 510 can evaluate data transfer or data copy operations involving a peripheral interface (e.g., a universal serial bus (USB) interface) of the computer 42 and/or the console 52.

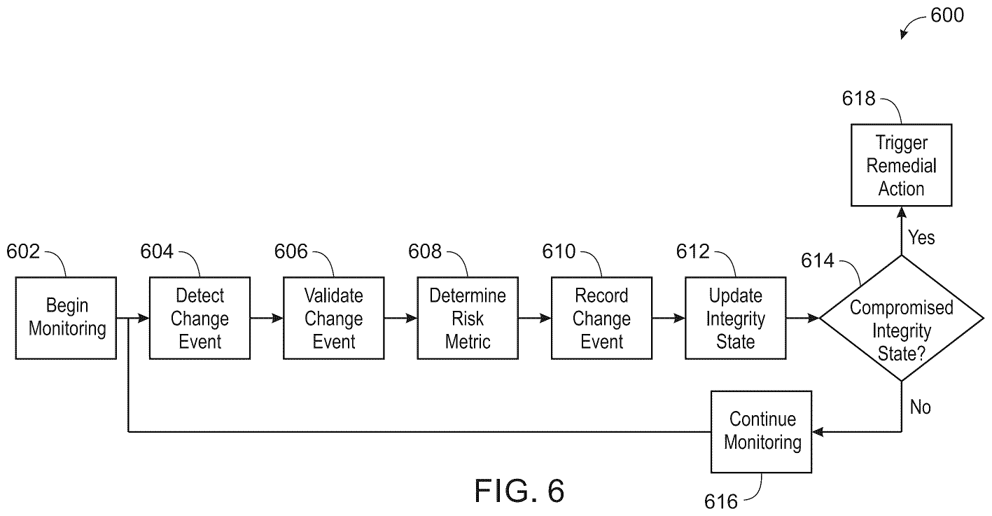

FIG. 6 is a flowchart of a method 600 of file integrity auditing for malware detection, in accordance with aspects of the present disclosure. The method 600 may be performed by one or more components (e.g., processing circuitry) of the CT imaging system 10 in FIGS. 1-3 or another type of medical imaging, monitoring, or therapeutic system. One or more steps of the method 600 may be performed simultaneously and/or in a different order than depicted in FIG. 6. At block 602, the method 600 includes begin monitoring file system data that characterizes a memory state of a computing device. For example, the computer 42 stores file system data that characterizes a memory state of the CT imaging system 10, as described above with respect to FIGS. 1-3. In certain embodiments, the file system data that characterizes the memory state of the computing device can be continuously monitored.

At block 604, the method 600 also includes detecting a change event that modifies the memory state of the computing device. Continuing with the previous example, the file system data of the computer 42 can include a registry file that defines a storage location for reconstructed images provided by the image reconstructor 48. At a first time, the registry file includes a first value that defines the mass storage device 50 as the storage location for reconstructed images provided by the image reconstructor 48. At a second time (subsequent to the first time), the registry file includes a second value that defines a different storage location for reconstructed images provided by the image reconstructor 48. Between the first time and the second time, a write file operation can be performed on the registry file to overwrite the first value with the second value. The write file operation performed on the registry file modifies registry file content by overwriting the first value with the second value. Modifying registry file content involves modifying the file system data of the computer 42 that includes the registry file. Modifying the file system data of the computer 42 involves modifying the memory state of the CT imaging system 10 that is characterized by the file system data of the computer 42. Overwriting the first value of the registry file with the second value can represent a change event (e.g., a registry overwrite event) that modifies the memory state of the CT imaging system 10.

In certain embodiments, detecting the change event can include accessing file data that characterizes the change event. Continuing with the registry overwrite example, the file system data of the computer 42 includes registry file content that changes between the first time and the second time. The registry overwrite event can be characterized by comparing registry file content at the first time with the registry file content at the second time. As described above with respect to FIGS. 1-3, the file system data of the computer 42 can include various types of file data, such as configuration data. Registry file content is one example of configuration data that can be included in the file system data of the computer 42. In certain embodiment, the file data that characterizes the change event can be accessed using an application programming interface (API) or a library provided by an operating system of the computing device. Continuing with the registry overwrite example, configuration data and other file system data of the computer 42 can be accessed by sending calls (e.g., system calls and/or library calls) to an API or a library provided by an operating system of the computer 42.

At block 606, the method 600 also includes validating the change event based on a comparison between the change event and a baseline representation of the memory state. Validating a change event generally involves evaluating consistency between the change event and a baseline representation. An inverse relationship can exist between that consistency and a likelihood that the change event is indicative of malicious behavior. For example, higher consistency between a change event and a baseline representation reduces a likelihood that the change event is indicative of malicious behavior. Conversely, lower consistency between a change event and a baseline representation increases a likelihood that the change event is indicative of malicious behavior.

As described above with respect to FIGS. 1-3, the computer 42 can include a baseline representation of the memory state of the CT imaging system 10. Continuing with the registry overwrite example, a comparison between the configuration data that characterizes the registry overwrite event and a baseline representation of the memory state of the CT imaging system 10 can validate the registry overwrite event. The comparison generally involves evaluating consistency between the registry overwrite event and the baseline representation. For example, the comparison can involve evaluating whether any modification of the registry file is consistent with the baseline representation. In another example, the comparison can involve evaluating whether the registry file having the second value is consistent with the baseline representation.

In certain embodiments, a comparison between a change event and a baseline representation can involve evaluating context data that characterizes operational conditions proximate to the change event. Continuing with the registry overwrite example, context data can include temporal data (e.g., date and/or time of the registry overwrite event), network data (e.g., availability of the gateway 306), external device data (e.g., operational state of the PACS 308), internal device data (e.g., operational state of a network interface of the computer 42), and other data that characterizes operational conditions proximate to the registry overwrite event.

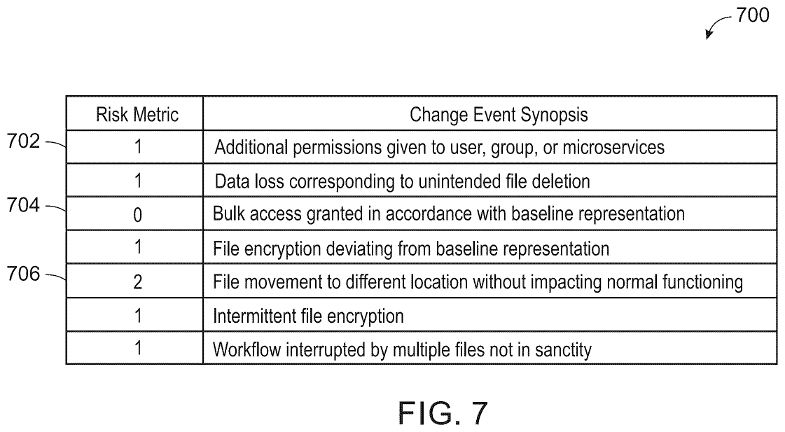

At block 608, the method 600 also includes determining a risk metric for the change event based on the comparison. Continuing with the registry overwrite example, a risk metric can be determined for the registry overwrite event based on the comparison between the configuration data and the baseline representation. Determining the risk metric for the registry overwrite event can involve evaluating a likelihood that malware operation initiated the registry overwrite event. Determining the risk metric for the registry overwrite event can also involve evaluating a severity of damage to the CT imaging system 10 (or similar processor-based system) and/or to the operational environment 300 if malware operation initiated the registry overwrite event. In certain embodiments, determining the risk metric can involve calculating a probability that estimates a likelihood that malware operation initiated the change event. In certain embodiments, determining the risk metric can involve assigning a discrete value or score to the change event based on a risk metric-change event association. The discrete value can be assigned from a set of discrete values with each discrete value representing a different level of risk. An example of assigning discrete values to change events is discussed below with respect to FIG. 7. In certain embodiments, risk metric-change event associations can be learned during the development process 400 discussed above with respect to FIG. 4.

At block 610, the method 600 also includes recording the change event to an event log file. Recording a change event to an event log file generally involves storing relevant information regarding the change event to facilitate subsequent analysis or self-learning. Relevant information regarding a change event can include: file data that characterizes the change event, context data that characterizes operational conditions proximate to the change event, a risk score determined for the change event, and the like. Continuing with the registry overwrite example, recording the registry overwrite event to an event log file can involve storing the configuration data that characterizes the registry overwrite event, the context data that characterizes operational conditions proximate to the registry overwrite event, the risk metric determined for the registry overwrite event, or a combination thereof.

At block 612, the method 600 also includes updating an integrity state of the computing device based on the comparison. Updating an integrity state of a computing device generally involves determining a likelihood of file integrity compromise based on a likelihood that a change event is indicative of malicious behavior. A direct relationship can exist between a likelihood of file integrity compromise and a likelihood that a change event is indicative of malicious behavior. For example, increasing likelihood that a change event is indicative of malicious behavior increases a likelihood of file integrity compromise. Conversely, decreasing likelihood that a change event is indicative of malicious behavior decreases a likelihood of file integrity compromise.

Continuing with the registry overwrite example, an integrity state of the CT imaging system 10 can be updated based on the comparison between the configuration data that characterizes the registry overwrite event and the baseline representation. If that comparison determines the registry overwrite event is not indicative of malicious behavior (e.g., the registry overwrite event is consistent with the baseline representation), the integrity state of the CT imaging system 10 (or other processor-based system) can be updated to a non-compromised integrity state. If that comparison determines the registry overwrite event is indicative of malicious behavior (e.g., the registry overwrite event is inconsistent with the baseline representation), the integrity state of the CT imaging system 10 can be updated to a compromised integrity state.

At block 614, the method 600 also includes determining whether the updated integrity state of the computing device is a compromised integrity state. If the updated integrity state of the computing device is not the compromised integrity state (e.g., the updated integrity state is a non-compromised integrity state), the method 600 also includes continuing to monitor the file system data that characterizes the memory state of the computing device, at block 616. Continuing with the registry overwrite example where the updated integrity state of the CT imaging system 10 is the non-compromised integrity state, file system data that characterizes a memory state of the CT imaging system 10 can continue to be monitored.

If the updated integrity state of the computing device is the compromised integrity state, the method 600 also includes triggering a remedial action (e.g., via control signals), at block 618. Triggering a remedial action can involve transmitting a notification regarding the change event. The notification can include various information regarding the change event, such as file data that characterizes the change event, context data that characterizes operational conditions proximate to the change event, a risk score determined for the change event, and the like. In certain embodiments, the notification can also include presenting an indicator (e.g., a warning icon) on a display (e.g., on a console for a medical imaging system) or modifying an existing indicator (e.g., transitioning an indicator color from green to red). The notification can be transmitted to a display associated with the computing device (e.g., a console for a medical imaging system) or another device (e.g., an administrator device). In certain embodiments, the notification can be transmitted when a risk metric determined for the change event exceeds a defined threshold. Triggering a remedial action can also involve automatically modifying operation of the computing device to mitigate further compromise of the computing device or other devices. Modifying operation of the computing device can involve terminating one or more network connections to isolate the computing device from external devices. Modifying operation of the computing device can also involve deactivating a functionality of the computing device, such as to ensure patient safety and/or to protect patient or medical data.

Continuing with the registry overwrite example where the updated integrity state of the CT imaging system 10 is the compromised integrity state, a notification can be transmitted to the display 54, to a display of an administrator device (not depicted) within the operational environment 300, or both. Additionally, or alternatively, operation of the CT imaging system 10 (or other processor-based system) can be automatically modified to mitigate further compromise of the CT imaging system 10, other devices in the operational environment 300, or both. For example, a network interface of the computer 42 can be automatically deactivated to isolate the CT imaging system 10 from other devices in the operational environment 300. In another example, a radiation exposure functionality of the CT imaging system 10 can be deactivated.

In certain embodiments, the change event can be initiated on another device that is communicatively coupled to the computing device via a network interface. In certain embodiments, validating the change event can involve providing file data that characterizes the change event as input to a classification model. In certain embodiments, the classification model can be trained using the baseline representation of the memory state. In certain embodiments, the change event corresponds to a conditional indicator of a conditional indicator set. For example, the change event and the baseline representation can have different file size values for a given file. In this example, the change event corresponds to a condition indicator that evaluates file-level variances.

In certain embodiments, where the change event corresponds to a conditional indicator of a conditional indicator set, the classification model can be trained using the conditional indicator set. In certain embodiments, where the change event corresponds to a conditional indicator set, the conditional indicator set can include a file indicator subset, a folder indicator subset, a process indicator subset, a prerequisite indicator subset, a peripheral device subset, or a combination thereof. In certain embodiments, the baseline representation of the memory state includes file data obtained from a file system in a reference configuration.

In certain embodiments, the event log file can store aggregated file data characterizing a plurality of detected change events. In certain embodiments where the event log file stores aggregated file data, a classification model can be updated using the event log file. For example, aggregated file data stored in the event log file can be provided as unlabeled input data to a machine learning process that employs unsupervised learning techniques (e.g., K-means, hierarchical clustering, mixture models, principal component analysis, autoencoders, and the like) to identify patterns within the aggregated file data. In this example, the machine learning process can provide output data that adjusts the baseline representation of the memory state (e.g., adjusts one or more values of the base line representation) and/or updates the conditional indicator set (e.g., adds a new conditional indicator to the conditional indicator set). In certain embodiments, the event log file may be stored locally in a memory of the medical imaging system or the facility having the medical imaging system. In certain embodiments, the event log file may be stored in a memory or database remotely located from the medical imaging system or the facility having the medical imaging system (e.g., manufacturer or provider of the medical imaging system).

FIG. 7 is a schematic diagram of an example error log data structure 700, in accordance with aspects of the present disclosure. The error log data structure 700 can include various information regarding a number of detected change events. In FIG. 7, the error log data structure 700 includes a change event synopsis for each detected change event. A change event synopsis can provide specific or general descriptive information regarding a corresponding change event. For example, the change event synopsis corresponding to a first change event 702 provides general descriptive information regarding the first change event 702. A change event synopsis can also provide results information regarding a comparison between a baseline representation and a corresponding change event. For example, the change event synopsis corresponding to a second change event 704 provides results information regarding a comparison between a baseline representation and the second change event 704. The change event synopsis corresponding to the second change event 704 also provides general descriptive information regarding the second change event 704. A change event synopsis can also provide operational impact information regarding any functionality changes associated with a corresponding change event. For example, the change event synopsis corresponding to a third change event 706 provides operational impact information associated with the third change event 706. The change event synopsis corresponding to the third change event 706 also provides general descriptive information regarding the third change event 706.

The error log data structure 700 also includes a risk metric determined for each detect change event. Each risk metric in the error log data structure 700 is a discrete value that is assigned to a corresponding change event based on risk metric-change event associations. The discrete value can be assigned from a set of discrete values with each discrete value representing a different level of risk. In FIG. 7, the set of discrete values includes a first discrete value (e.g., “0”) that represents a low level of risk, a second discrete value (e.g., “2”) that represents a medium level of risk, and a third discrete value (e.g., “1”) that represents a high level of risk. The second change event 704 is associated with the first discrete value (e.g., “0”) score in the error log data structure 700 to illustrate that error log data structures can include information regarding each detected change event. The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A medical imaging device comprising:
memory storing instructions that include a classification model; and
a processor communicatively coupled to the memory, wherein the instructions, when executed by the processor, cause the medical imaging device to:
implement an imaging-related functionality;
detect a change event that modifies a state of the memory;
validate the change event based on a comparison between the change event and a baseline representation of the state; and
update an integrity state of the medical imaging device based on the comparison.

2. The medical imaging device of claim 1, wherein the instructions, when executed by the processor, cause the medical imaging device to implement the imaging-related functionality by generating image data.

3. The medical imaging device of claim 1, wherein the change event modifies a digital imaging and communications in medicine (DICOM) file stored in the memory.

4. A computer-implemented method comprising:
detecting, by a processor of a medical imaging device, a change event that modifies a memory state of the medical imaging device;
validating, by the processor, the change event based on a comparison between the change event and a baseline representation of the memory state; and
updating, by the processor, an integrity state of the medical imaging device based on the comparison.

5. The computer-implemented method of claim 4, wherein detecting the change event comprises continuously monitoring file system data that characterizes the memory state of the medical imaging device.

6. The computer-implemented method of claim 4, wherein validating the change event comprises providing file data that characterizes the change event as an input to a classification model.

7. The computer-implemented method of claim 6, wherein the classification model is trained using the baseline representation of the memory state.

8. The computer-implemented method of claim 6, wherein validating the change event comprises receiving a malicious classification or a benign classification as an output of the classification model based on the file data that characterizes the change event and a similarity measurement.

9. The computer-implemented method of claim 6, wherein the classification model is included in a self-contained application package that is stored in a memory of the medical imaging device.

10. The computer-implemented method of claim 4, wherein the baseline representation of the memory state includes file data obtained from a file system in a reference configuration.

11. The computer-implemented method of claim 4, further comprising:
determining, by the processor, a risk metric for the change event based on the comparison, wherein the risk metric estimates a likelihood that malware operation initiated the change event.

12. The computer-implemented method of claim 11, further comprising:
transmitting, by the processor, a notification to an administrator device when the risk metric exceeds a defined threshold.

13. The computer-implemented method of claim 4, wherein detecting the change event comprises accessing file data that characterizes the change event using an application programming interface or a library provided by an operating system of the medical imaging device.

14. The computer-implemented method of claim 4, further comprising:
triggering, by the processor, a remedial action when the updated integrity state is a compromised integrity state.

15. The computer-implemented method of claim 4, further comprising:
recording, by the processor, file data that characterizes the change event to an event log file.

16. The computer-implemented method of claim 4, further comprising:
updating, by the processor, a classification model using an event log file that stores aggregated file data characterizing a plurality of detected change events.

17. The computer-implemented method of claim 4, wherein the change event corresponds to a conditional indicator of a conditional indicator set, and a classification model is trained using the conditional indicator set.

18. The computer-implemented method of claim 17, wherein the conditional indicator set includes a file indicator subset, a folder indicator subset, a process indicator subset, a prerequisite indicator subset, and/or a peripheral device subset, or a combination thereof.

19. The computer-implemented method of claim 4, wherein the change event is initiated on another device that is communicatively coupled to the medical imaging device via a network interface.

20. A non-transitory computer-readable medium, the computer-readable medium comprising processor-executable code that when executed by a processor, causes the processor to:
detect a change event that modifies a memory state of a computing device;
provide file data characterizing the change event as input to a classification model that is trained using a baseline representation of the memory state;
receive a classification and a risk score for the change event at an output of the classification model; and
update an integrity state of the computing device based on the classification and the risk score.
